(19) United States
(12) Patent Application Publication
(10) Pub. No.: US 2011/0009596 A1
(43) Pub. Date: Jan. 13, 2011

(54) PROCESS FOR THE PREPARATION OF N-PROTECTED-DECYLAMINOETHANAL

(75) Inventors: Ettore Bigatti, Rho (IT); Deborah Bollini, Covo (IT); Augusto Canavesi, Locate Varesino (CO) (IT); Ondrej Simo, Trnava (SK)

Correspondence Address: MERCHANT & GOULD PC, P.O. BOX 2903, MINNEAPOLIS, MN, 55402-0903, US

(73) Assignee: Plus Chemicals SA, Paradiso (CH)

(21) Appl. No.: 12/832,677

(22) Filed: Jul. 8, 2010

Related U.S. Application Data
(60) Provisional application No. 61/224,178, filed on Jul. 9, 2009.
(60) Provisional application No. 61/231,465, filed on Aug. 5, 2009.

(51) Int. Cl. C07K 9/00; C07C 235/34; C07D 233/02

(52) U.S. Cl. 530/322; 564/134; 548/300.1

(57) ABSTRACT

Compounds useful in the preparation of telavancin, for example, were prepared. These compounds include decylaminoethanal dialkyl acetals and N-protected decylaminoethanal dialkyl acetals, imidazolidine derivatives, and N-protected-decylaminoethanal.

### CROSS-REFERENCE TO RELATED APPLICATIONS

This application claims the benefit of U.S. Provisional Patent Application Ser. Nos. 61/224,178, filed Jul. 9, 2009 and 61/231,465, filed Aug. 5, 2009, which are incorporated herein by reference.

### FIELD OF INVENTION

The invention relates to processes for preparing N-protected-decylaminoethanal and to synthetic intermediates, which are useful, for example, in the preparation of telavancin.

### BACKGROUND OF THE INVENTION

Telavancin, N3″-[2-(decylamino)ethyl]-29-[[(phosphonomethyl)amino]-methyl]-vancomycin hydrochloride of the following formula:

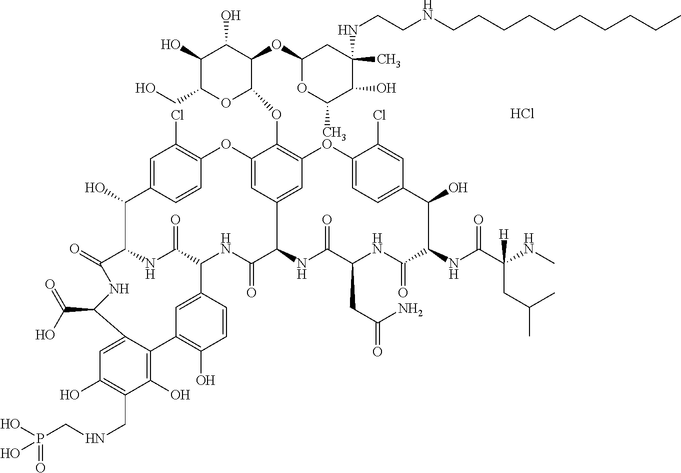

is a bactericidal phosphonate derivative of lipoglycopeptide antibiotic Vancomycin indicated for the treatment of Gram-positive bacterial infection.

Telavancin is marketed under the trade name VIBATIV™ by Astellas Pharma US. It was approved by the FDA in October 2007.

Telavancin and its preparation are disclosed in U.S. Pat. No. 6,635,618. There, Telavancin is prepared by reacting N-protected decylaminoethanal of formula IV with Vancomycin, of formula V (scheme 1), leading, after deprotection, to the intermediate of formula VII. This intermediate is then phosphonated on the 1,3-dihydroxyphenyl moiety, providing Telavancin.

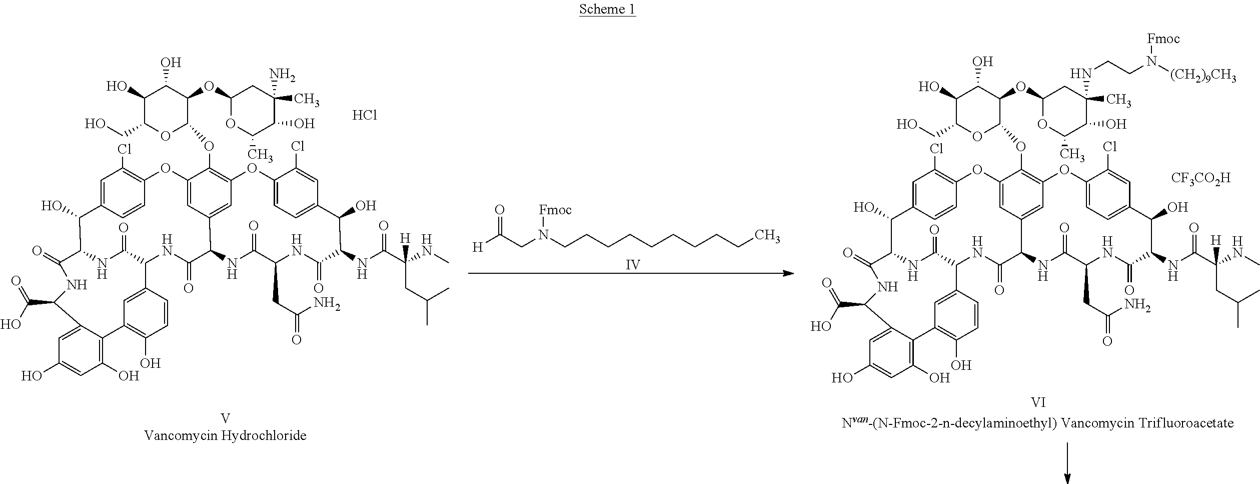

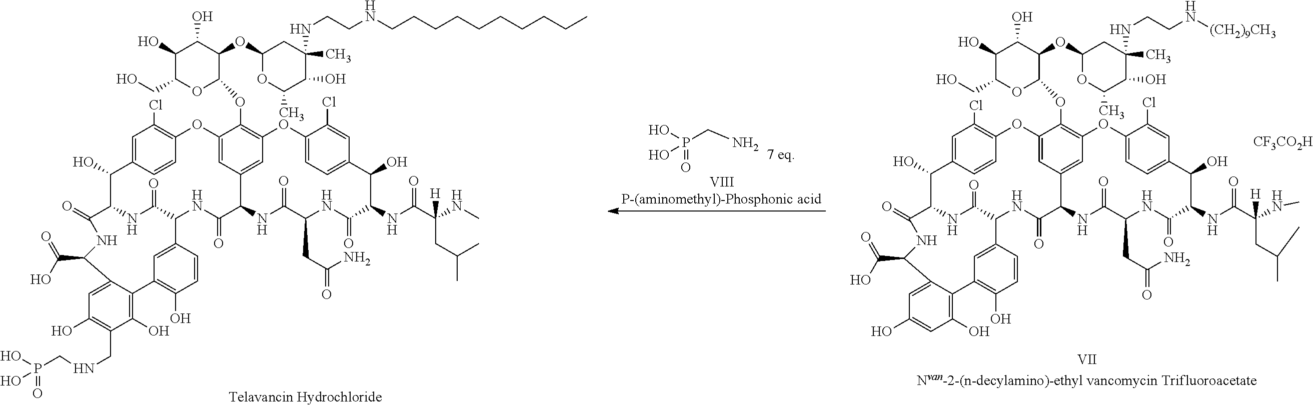

The N-protected decylaminoethanal of formula IV is prepared by oxidation of formula III (scheme 2B) according to the process reported in U.S. Pat. No. 6,635,618. This oxidation is performed at a temperature of about −40° C., probably to avoid oxidation of the aldehyde to carboxylic acid, which is a side-reaction. In addition, the oxidation also includes the use of the toxic reagent oxalyl chloride.

N-decylaminoethanol of formula I, which is the starting material for the preparation of N-protected decylaminoethanal of formula IV, can be prepared according to the process reported in DE4215559 (scheme 2A). There, the imine intermediate undergoes hydrogenation at a temperature of 160° C. and at the pressure of 130 Bar, providing the N-decylaminoethanol.

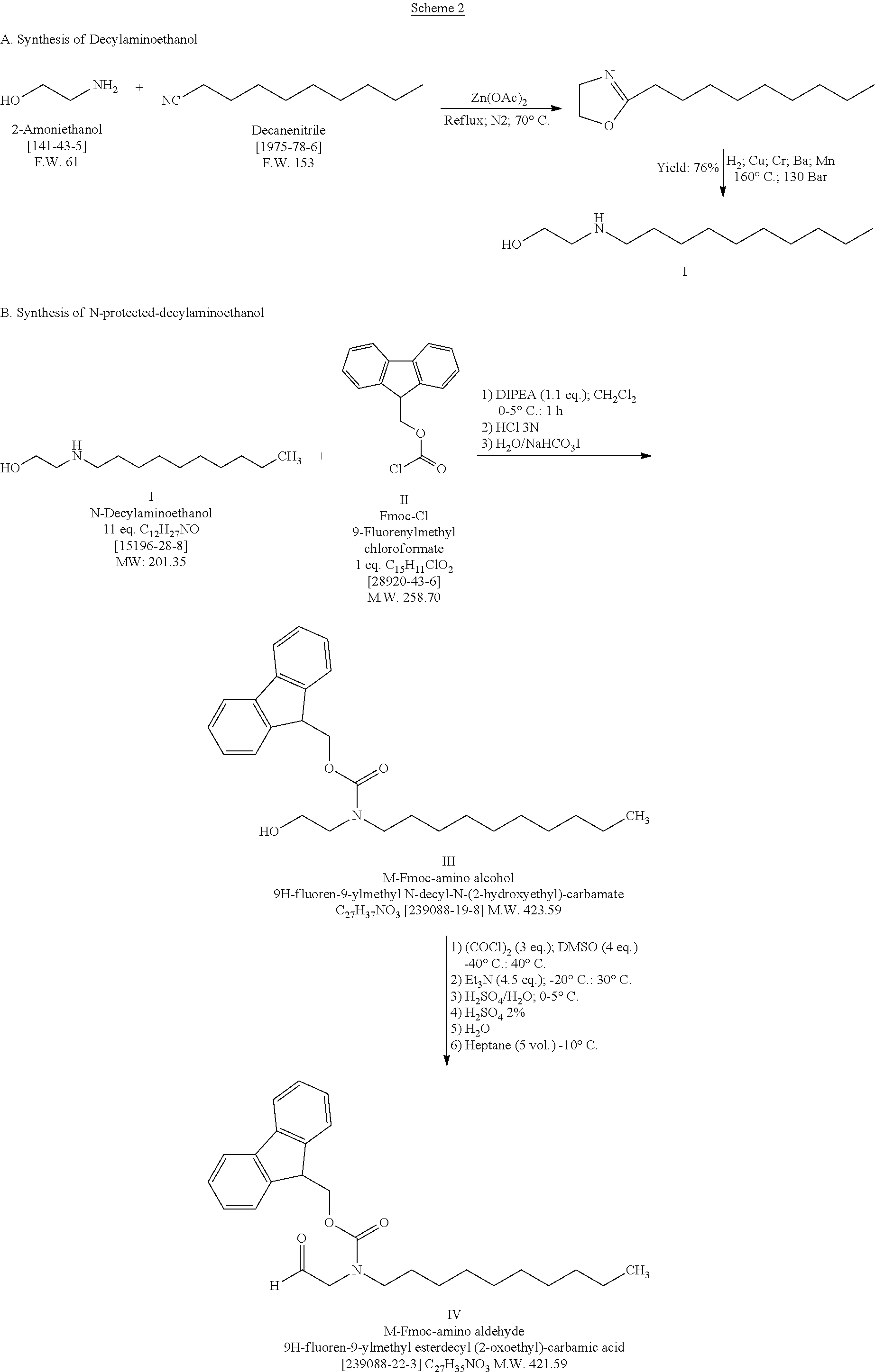

Thus, there is a need in the art for new and efficient processes for the preparation of the N-protected-decylaminoethanal that use less harsh conditions and reagents and are more suitable for industrial scale.

### SUMMARY OF THE INVENTION

According to one embodiment, the present invention comprises a process for preparing N-protected-decylaminoethanal comprising
a) reacting i) glyoxal 1,1 dialkyl acetal and decylamine or

ii) aminoacetaldehyde dialkyl acetal and decanal to provide an imine intermediate and reducing the imine intermediate with a reducing agent to obtain decylaminoethanal dialkyl acetal of formula III,

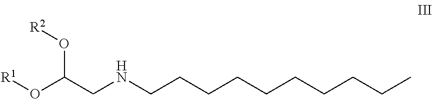

b) reacting the compound of formula III with an amine protecting group donor to obtain an N-protected decylaminoethanal dialkyl acetal of formula IV, and

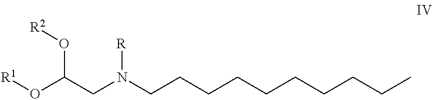

c) reacting the N-protected-decylaminoethanal dialkyl acetal of formula IV with an acid to obtain N-protected-decylaminoethanal of formula V,

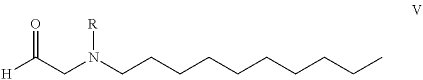

wherein, R is an amine protecting group, such as 9-Fluorenylmethyl Carbamate (“Fmoc”), formyl, acetyl, trifluoroacetyl, benzyl, benzyloxycarbonyl (“Cbz”), t-butoxycarbonyl (“BOC”), trimethylsilyl (“TMS”), 2-trimethylsilylethanesulfonyl, (“SES”), trityl and substituted trityl groups, allyloxycarbonyl, nitroveratryloxycarbonyl (“NVOC”), and allyloxycarbonyl (Alloc) and R1 and R2 are independently selected from C1-C3 alkyl or combined to form a 5- or 6-member cyclic acetal ring.

In another embodiment, the present invention comprises decylaminoethanal dialkyl acetal of formula III:

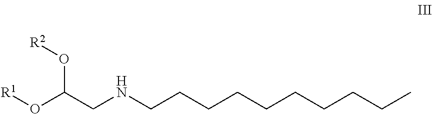

wherein, R1 and R2 are independently selected from C1-C3 alkyl or combined to form a 5- or 6-member cyclic acetal ring.

In another embodiment, the present invention comprises a process of preparing decylaminoethanal dialkyl acetal of formula III comprising reacting

i) glyoxal 1,1 dialkyl acetal and decylamine, or

ii) aminoacetaldehyde dialkyl acetal and decanal to provide an imine intermediate and reducing the imine intermediate with a reducing agent.

In yet another embodiment, the present invention comprises a process for preparing Telavancin comprising preparing the compound of formula III according to the process of the present invention and converting it to Telavancin.

In yet another embodiment, the present invention comprises the use of the compound of formula III for the preparation of Telavancin.

In one embodiment, the present invention comprises N-protected-decylaminoethanal dialkyl acetal of formula IV

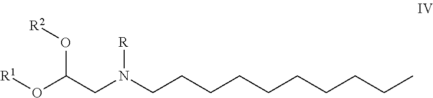

In another embodiment, the present invention comprises a process of preparing N-protected-decylaminoethanal dialkyl acetal of formula IV comprising reacting the compound of formula III with an amine protecting group donor, wherein R is as 9-Fluorenylmethyl Carbamate (“Fmoc”), formyl, acetyl, trifluoroacetyl, benzyl, benzyloxycarbonyl (“Cbz”), t-butoxycarbonyl (“BOC”), trimethylsilyl (“TMS”), 2-trimethylsilylethanesulfonyl, (“SES”), trityl and substituted trityl groups, allyloxycarbonyl, nitroveratryloxycarbonyl (“NVOC”), and allyloxycarbonyl (Alloc) and R1 and R2 are independently selected from C1-C3 alkyl or combined to form a 5- or 6-member cyclic acetal ring.

In yet another embodiment, the present invention comprises a process for preparing Telavancin comprising preparing the compound of formula IV according to the process of the present invention and converting it to Telavancin.

In yet another embodiment, the present invention comprises the use of the compound of formula IV for the preparation of Telavancin.

In another embodiment, the present invention comprises a process of preparing N-protected-decylaminoethanal of formula V

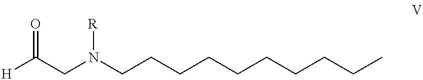

comprising removal of the acetal group from the compound of formula IV, preferably by reacting the compound of formula IV with an acid.

In yet another embodiment, the present invention comprises a process for preparing Telavancin comprising preparing the compound of formula V according to the process of the present invention and converting it to Telavancin.

In one embodiment, the present invention comprises the imidazolidine derivative of formula V′

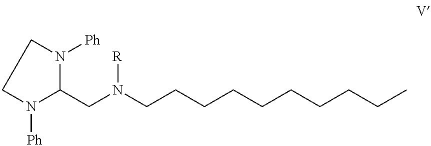

In another embodiment, the present invention comprises a process for preparing N-protected-decylaminoethanal of formula V comprising preparing the imidazolidine derivative of formula V′ and converting it to N-protected-decylaminoethanal of formula V.

In yet another embodiment, the present invention comprises a process for preparing Telavancin comprising preparing the imidazolidine derivative of formula V′ and converting it to Telavancin.

In yet another embodiment, the present invention comprises the use of the compound of formula V′ for the preparation of Telavancin.

### DETAILED DESCRIPTION OF THE INVENTION

A thing, e.g., a reaction mixture, may be characterized herein as being at, or allowed to come to “room temperature.” This expression means that the temperature of the thing is close to, or the same as, that of the space, e.g., the room or fume hood, in which the thing is located. Typically, room temperature is from about 20° C. to about 30° C., or about 25° C.

A process or portion thereof may be referred to herein as being carried out “overnight.” This term refers to a time interval, e.g., for carrying out the process portion thereof, that spans the time during the night, when that process or step may not be actively observed. This time interval is from about 8 to about 20 hours, typically about 16 hours.

As used herein, the term “amine protecting group”, e.g., “nitrogen protecting group”, relates to a molecule that contains a group which, when bound to an amino group of the compound, prevents undesired reactions from occurring at this amino group and which can be removed by conventional chemical or enzymatic steps to reestablish the amino group, e.g., 9-Fluorenylmethyl Carbamate (“Fmoc”), formyl, acetyl, trifluoroacetyl, benzyl, benzyloxycarbonyl (“Cbz”), t-butoxycarbonyl (“BOC”), trimethylsilyl (“TMS”), 2-trimethylsilylethanesulfonyl, (“SES”), trityl and substituted trityl groups, allyloxycarbonyl, nitroveratryloxycarbonyl (“NVOC”), and allyloxycarbonyl (Alloc).

The present invention offers new synthetic pathways for preparing N-protected-decylaminoethanal. Preferred routes of synthesis can be illustrated by the following schemes:

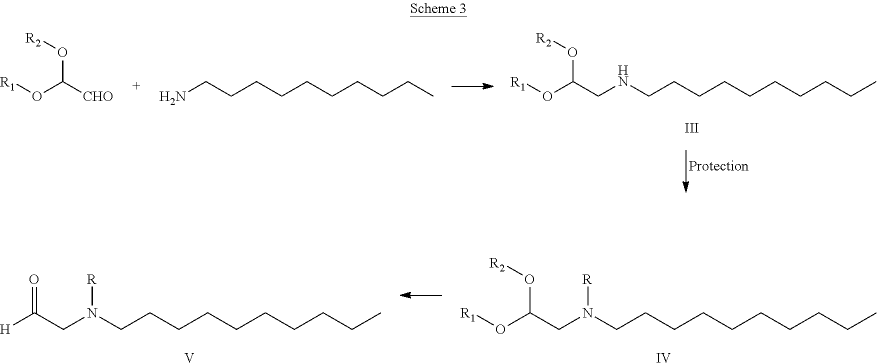

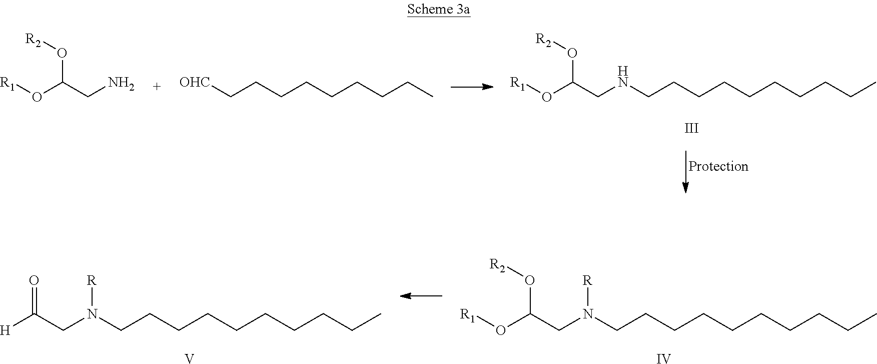

The above process provides N-protected-decylaminoethanal of formula V

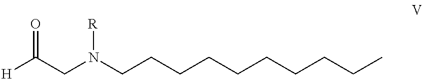

in high yield and purity in a one pot manner.

The above processes proceed via novel synthetic intermediates, including intermediates of the following formulae:

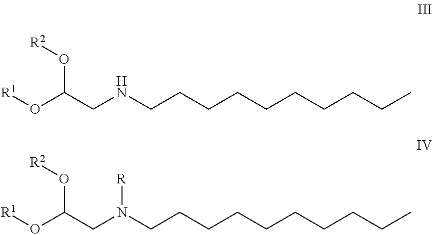

The present invention encompasses these intermediates, as well as their use in a process for the manufacture of vancomycin derivatives, in particular telavancin.

The formula III compound obtained via this process is already at the aldehyde oxidation state. Thus, there is no need for an oxidation step as described in the prior art.

Avoiding the oxidation step provides a superior process in light of the following:
The protecting groups that can be used are not restricted to groups that should be stable under harsh oxidation conditions. This enables flexibility in choosing the protecting group based on other factors, such as safety and cost.
It is possible to obtain the compound of formula V via a hydrolysis step that requires mild conditions.
Performing the hydrolyzing of the dialkyl acetal protecting groups according to the process of the present invention allows the isolation of the final product in a straightforward manner. In contrast, a previously reported route of synthesis includes the difficult isolation of the compound of formula IV, as described in scheme 2, due to the formation of a complex reaction mixture in the oxidation step.

In addition, the process for providing the new dialkyl acetal of formula III can be done in a one pot manner.

The above process for preparing N-protected-decylaminoethanal of formula V comprises:
a) reacting i) glyoxal 1, 1 dialkyl acetal and decylamine, or

ii) aminoacetaldehyde dialkyl acetal and decanal to provide an imine intermediate and reducing the imine intermediate with a reducing agent to obtain decylaminoethanal dialkyl acetal of formula III,

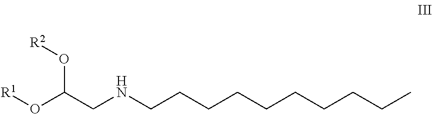

b) reacting the compound of formula III with an amine protecting group donor to obtain N-protected-decylaminoethanal dialkyl acetal of formula IV, and

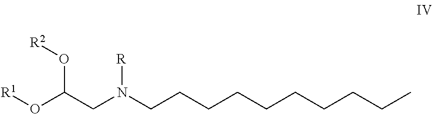

c) reacting N-protected-decylaminoethanal dialkyl acetal of formula IV with an acid to obtain N-protected-decylaminoethanal of formula V

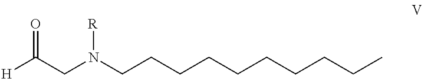

wherein R is an amine protecting group and R1 and R2 are independently selected from C1-C3 alkyl or combined to form a 5- or 6-member cyclic acetal ring.

Step a) of the above process can be done in a one pot manner.

The imine intermediate can be described by the following formula:

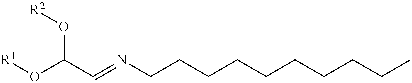

The compound of Formula III can be prepared from the compounds of i) glyoxal 1,1 dialkyl acetal and decylamine, or ii) aminoacetaldehyde dialkyl acetal and decanal, in a “one-pot process”. The term refers to a process in which the intermediate, in this case the imine intermediate, is not separated from the reaction vessel. However, “one-pot processes” do not necessarily exclude steps entailing the separation and/or isolation of substances other than the imine intermediate.

Examples of suitable reducing agents are sodium cyanoborohydride, sodium triacetoxyborohydride, pyridine/borane, sodium borohydride, zinc borohydride and molecular hydrogen in the presence of a hydrogenation catalyst.

Suitable dialkyl acetal include, for example, dimethyl acetal, diethyl acetal or cyclic acetal such as 5-Methylene-1,3-dioxane, 5,5-Dibromo-1,3-dioxane, 5-(2′-Pyridyl)-1,3-dioxane, 5-TrimethylsilyI-1,3-dioxane, 4-Bromomethyl-1,3-dioxolane, 4-(3-Butenyl)-1,3-dioxolane, 4-Phenyl-1,3-dioxolane, 4-(4-Methoxyphenyl)-1,3-dioxolane or 4-Trimethylsilylmethyl-1,3-dioxolane.

According to an embodiment of the invention, when the dialkyl acetal is dimethyl acetal, decylamine and glyoxal 1,1-dimethylacetal or aminoacetaldehyde dimethyl acetal and decanal, are combined preferably with a polar organic solvent to form a reaction mixture leading to the in situ formation of the corresponding imine. This coupling step can be followed by a hydrogenation step.

The coupling and hydrogenation reactions lead to the formation of a new intermediate, decylaminoethanal dimethylacetal of formula III.

The polar organic solvent can be a protic organic solvent or a polar aprotic organic solvent. Suitable protic organic solvents can be alcohols such as C₁-C₄ alcohol, more preferably, methanol or butanol, most preferably methanol.

Suitable polar aprotic organic solvents include, for example, dipolar aprotic solvents such as dimethylformamide (DMF), dimethylacetamide (DMA), N-methyl-2-pyrrolidone (NMP), dimethyl sulfoxide (DMSO), 1,3-dimethyl-3,4,5,6-tetrahydro-2(1H)-pyrimidinone (DMPU), 1,3-dimethyl-2-imidazolidinone (DMI) and acetonitrile, preferably, DMF.

The glyoxal 1,1-dimethylacetal can be added, for example, as a 60% w/w solution in water.

When the solvent is butanol, the obtained reaction mixture can be distilled to remove the water formed in the reaction, in order to drive to completion the formation of the imine intermediate. The distillation can be carried out several times during the course of the reaction, or it can be carried out continuously, e.g., using a Dean-Stark trap or similar apparatus.

The distillation can be done under vacuum at a temperature of about 50° C. to 110° C., for example at about 60° C. to about 90° C., for example, at about 60° C.

The advancement of the reaction can be monitored, e.g., by gas chromatography (GC), for example by monitoring the changes in the amount of the imine intermediate.

The imine intermediate can then be reduced in situ by using reducing agent to give the amine of formula III.

Suitable reducing agents include, for example, sodium cyanoborohydride, sodium triacetoxyborohydride, pyridine/borane, sodium borohydride, zinc borohydride and molecular hydrogen in the presence of a hydrogenation catalyst.

Suitable hydrogenation catalysts include, for example, platinum, platinum on carbon, palladium on carbon, or nickel.

The catalyst can be used in an effective amount, for example in an amount of about 1% to about 10% w/w or of 1% w/w, in relation to the weight amount of decylamine or decanal.

The obtained reaction mixture can be further maintained to push the formation of the compound of formula III closer to completion. The reaction mixture can be maintained at a temperature of about 25° C. to about 70° C., for example at about 50° C., for a period of about 8 to about 16 hours, or about overnight when the solvent is butanol; and at a temperature of about 50° C. under a pressure of about 5 atm for a period of about 3 to about 16 hours, or about 3.5 hours when the solvent is methanol.

The progress of the reaction can be monitored, for example, by monitoring the amount of the product amine with GC. Based on the results, the reaction can be stopped, for example, by filtering the catalyst. Preferably, after filtering the catalyst, the obtained filtrate is evaporated, preferably, by distilling the solvent under vacuum at about 60° C., forming an oily residue containing decylaminoethanal dimethylacetal of formula III.

The obtained compound of formula III can be further purified by any suitable technique. For example, decylaminoethanal dimethylacetal of formula III can be converted to a suitable salt, e.g., a p-toluenesulfonic acid (“PTSA”) salt. The conversion to a salt can be done, for example, by dissolving the compound of formula III in a suitable solvent, e.g., toluene and adding PTSA to the solution.

The obtained compound of formula III can then be used to prepare the N-protected-decylaminoethanal dimethylacetal of formula IV.

The process for preparing the compound of formula IV comprises reacting the compound of formula III with an amine protecting group donor.

When the amine protecting group is benzyloxycarbonyl, the oily residue containing the compound of formula III can be combined with a suitable solvent to obtain a solution which is then cooled.

Suitable solvents include, for example, halogenated hydrocarbons (e.g., DCM), linear or branched ethers (e.g., MTBE), alcohols (e.g., MeOH, EtOH), DMSO, ACN or aromatic hydrocarbons (e.g., toluene).

The cooling can be done to a temperature of about −5° C. to about 5° C. The protecting group donor and an organic base are then added to the cooled solution.

The protecting group donor can be, for example, benzylchloroformate.

The protecting group donor can be used in an amount of about 1 to about 2 mole equivalents, or of about 1.1 to about 1.5 mole equivalents based on the amount of the compound of formula III, for example, about 1.0 to about 1.2 mole equivalents.

The organic base can be used in an amount of about 1.2 mole equivalents based on the amount of the compound of formula III are added.

A suitable organic base can be a C₆-C₈ amine, for example, diisopropylethylamine.

The reaction mixture can be maintained at a temperature of about 5° C. for a period of about 2 hours followed by further maintaining at about room temperature for about 3 hours.

The reaction can be stopped, for example, by washing the reaction mixture with water. Preferably, after washing, the organic layer is extracted and the extract then distilled under vacuum preferably at a temperature of about 35° C. forming an oily residue, containing N-protected-decylaminoethanolacetal of formula IV.

When the amine protecting group is Fmoc, the oily residue containing the compound of formula III can be dissolved in a suitable solvent, for example, toluene, to obtain a solution which is then cooled.

The cooling can be done to a temperature of about −5° C. to about 5° C. The protecting group donor and an organic base are then added to the cooled solution.

The protecting group donor can be Fmoc-chloride.

The protecting group donor can be used in an amount of about 1 to about 2 mole equivalents, or of about 1.1 to about 1.5 mole equivalents based on the amount of the compound of formula III, for example, of about 1.0 to about 1.1 mole equivalents.

The organic base can be used in an amount of about 1.2 mole equivalents based on the amount of the compound of formula III are added.

A suitable organic base can be sodium hydroxide or potassium hydroxide.

The reaction mixture can be maintained at a temperature of about 0° C. to about 5° C. for a period of 30 to about 60 minutes, for example for about 40 minutes, followed by further maintaining at about room temperature for about 20 minutes to form a two phase reaction mixture.

The product recovery may be done, for example, by separating the organic layer from the two phase reaction mixture. The organic layer can then be washed with water and distilled under vacuum preferably at a temperature of about 35° C. forming an oily residue.

The Formula IV compound can then be used for the preparation of a compound of formula V. The process for preparing a formula V compound comprises hydrolyzing the N-protected-decylaminoethanal dialkyl acetal of formula IV to obtain an N-protected-decylaminoethanal of formula V,

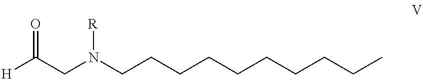

using an acid, wherein R is an amine protecting group.

The hydrolysis can be done by combining the obtained oily residue containing the compound of formula IV with a C₁-C₃ ketone and treating the mixture with an acid to form a two phase reaction mixture. A suitable C₁-C₃ ketone includes, for example, acetone.

The acid can be a mineral acid, such as HCl, formic acid, acetic acid and PTSA.

The reaction progress can be monitored, for example, using TLC, and the obtained compound of formula V can be recovered from the reaction mixture when the reaction is complete or has progressed to an acceptable degree.

When the acid is HCl, the product recovery may be done, for example, by separating the two phases reaction mixture comprising an organic phase and an aqueous phase that is obtained, and further distilling the upper/organic phase, preferably under vacuum at about 35° C., leading to the formation of oil, containing the N-protected-decylaminoethanal of formula V.

When the acid is formic acid, the product recovery may be done, for example, by adding an organic solvent to the reaction mixture, washing the reaction mixture with water and separating the organic layer from the two phases reaction mixture to obtain an oily residue.

The obtained compound of formula V can then be used for the preparation of Telavancin.

The present invention provides additional synthesis routes, described in the following scheme:

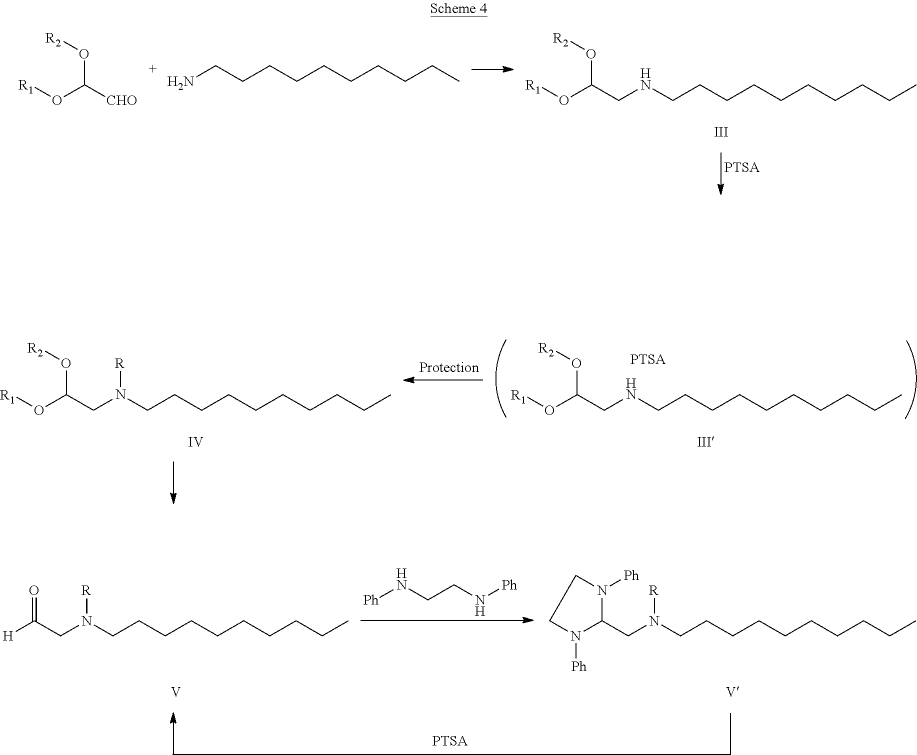

The above process further comprises two additional steps when compared to the process described earlier: conversion of the compound of formula III to a suitable salt, e.g., a PTSA salt of formula III′

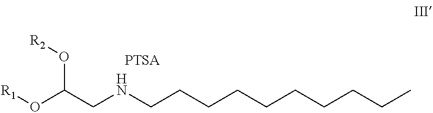

and providing imidazolidine derivative of formula V′ as a synthetic intermediate:

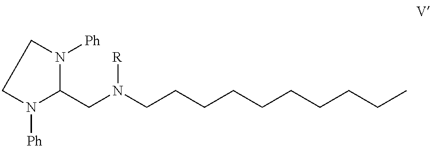

The present invention encompasses these intermediates, as well as their use in a process for the manufacture of vancomycin derivatives, in particular telavancin.

The PTSA salt of formula III′ can be prepared by providing a solution of the compound of formula III and PTSA in a suitable solvent, e.g., toluene. Then the solvent can be removed by evaporation under reduced pressure, e.g, from about 20 to about 70 mm Hg at a temperature of about 40° C. to about 50° C.

The product of Formula III′ can then be recovered. The recovery of formula III′ may be done by any suitable technique, for example by precipitation, filtering the suspension and washing the precipitate and drying. Washing can be done for example with a hydrocarbon solvent, e.g., n-heptane.

The Formula III′ compound can then be converted to a compound of formula IV. The process for the preparation of a compound of formula IV comprises reacting the compound of formula III′ with an amine protecting group donor in a suitable solvent, e.g., toluene.

A solution of the formula III′ compound in a suitable solvent, e.g., toluene, is cooled, e.g., to a temperature of about 0° C. to about 5° C., and then the amine protecting group donor and a base are added to the cooled solution.

A suitable organic base can be sodium hydroxide or potassium hydroxide.

The amine protecting group donor can be added to the reaction by first providing a solution of amine protecting group donor in a suitable solvent, e.g., toluene and then adding the obtained solution to the reaction.

The reaction mixture can be maintained at a temperature of about 0° C. to about 5° C. for a period of about 40 minutes followed by further maintaining at about room temperature for about 20 minutes to form a two phase reaction mixture.

The product recovery may be done, for example, by adding an organic solvent to the reaction mixture, washing the reaction mixture with water and separating the organic layer from the two phase reaction mixture to obtain an oily residue.

The Formula IV compound can then be converted to a compound of formula V′. The process for the preparation of the compound of formula V′ comprises reacting the compound of formula IV with an acid.

The process for the preparation of a formula V′ compound comprises a) combining the N-protected-decylaminoethanal dialkyl acetal of formula IV with an acid to obtain a two-phase reaction mixture b) adding a solvent and water to obtain two-phase mixture; c) separating the organic layer of the reaction mixture; d) combining the organic layer with 1, 2 dianilinoethane to obtain a solution and e) precipitating an imidazolidine compound of formula V′ from the solution.

The product of Formula V′ can then be recovered by any suitable technique, for example by precipitation, separating the precipitate by filtration and washing the filtered precipitate and drying it. Washing can be done for example with acetone.

The obtained compound of formula V′ can be further purified by any suitable technique. For example, the compound of formula V′ can be converted to a suitable salt, e.g., a p-toluenesulfonic acid (“PTSA”) salt.

The Formula V′ compound can then be converted to a compound of formula V. The process for the preparation of a formula V compound comprises dissolving the compound of formula V′ in a suitable solvent, e.g., tetrahydrofuran (“THF”) and adding PTSA to the solution. The solution can be provided by combining the compound of formula V′ and THF and heating the combination to a temperature of about 30° C. to about 40° C. The solution formed thereby is then cooled to a temperature of about 15° C. to about 20° C. The cooled solution is then reacted with a solution of PTSA in THF. The reaction mixture can be further maintained at the same temperature for a period of about 20 minutes.

The product of Formula V can then be recovered by any suitable technique, for example by precipitation, filtering the suspension and washing the precipitate and drying it. Washing can be done for example with concentrated sodium bicarbonate and water.

The step of providing the compound of formula V′ not only allows the purification of formula V but also provide a solid material, which is in contrast to other steps where an oily residue is obtained.

In another embodiment the present invention further provides another synthesis route for preparing the compound of formula V, described in the following scheme:

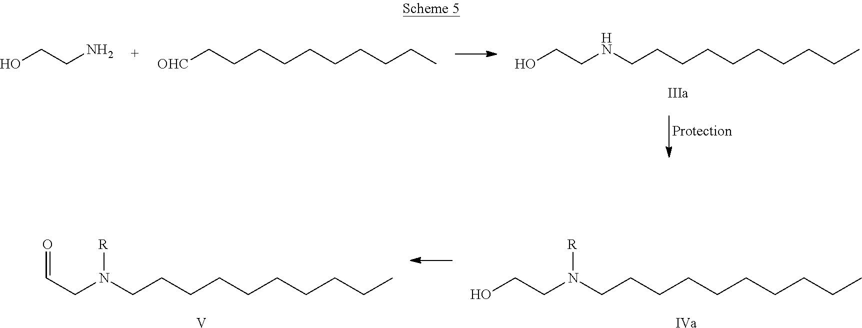

The hydrogenation step in the above process can be performed under mild conditions e.g., at room temperature and at a pressure of about 2 Bar. These conditions are suitable for industrial production.

In addition, the process according to scheme 5 can be done in a one-pot manner, without the need to isolate the intermediate of formula IIIa

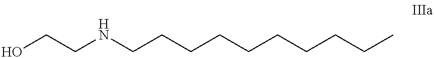

The above process comprises preparing decylaminoethanol of formula IIIa, said process comprising reacting decanal with ethanolamine and molecular hydrogen in the presence of a suitable hydrogenation catalyst.

Decanal, a natural product, and ethanolamine are reacted in a polar organic solvent, to form the corresponding imine. The polar organic solvent is a protic organic solvent, or a polar aprotic organic solvent. Suitable protic organic solvents can be alcohols such as C₁-C₄ alcohol, more preferably, butanol.

Suitable polar aprotic organic solvents can be dipolar aprotic solvents such as dimethylformamide (DMF), dimethylacetamide (DMA), N-methyl-2-pyrrolidone (NMP), dimethyl sulfoxide (DMSO), 1,3-dimethyl-3,4,5,6-tetrahydro-2(1H)-pyrimidinone (DMPU), 1,3-dimethyl-2-imidazolidinone (DMI), tetrahydrofurane (THF), toluene and acetonitrile, preferably, DMF.

The obtained imine is reacted in situ with molecular hydrogen in the presence of a suitable hydrogenation catalyst, for example Platinum on carbon.

The catalyst can be used in an effective amount, e.g., about 2% to about 5% w/w, for example of about 2% in relation to the mole amount of the imine. The reaction can be performed at about room temperature and can be maintained for overnight.

The obtained compound of formula Ma can then be recovered from the reaction mixture, if required. The reaction can be stopped, for example, by filtering the catalyst and washing the panel with a solvent, for example, the same solvent used before. Then, the solvent in the filtrate can be removed, preferably by distillation under vacuum at a temperature of about 35° C. to about 40° C. leading to the formation of a first oil, containing the compound of formula IIIa.

The desired product can be recovered from the first oil, for example, by combining the first oil with water and extracting the mixture with a C₁-C₂ halogenated hydrocarbon; the obtained organic layers are combined and the solvent is removed by distillation preferably under vacuum at about 35° C. leading the formation of a second oil, containing the compound of formula IIIa. The C₁-C₂ halogenated hydrocarbon can be for example dichloromethane (DCM).

After the formation of decylaminoethanol, the nitrogen group is protected with a protecting group. Then the protected product is oxidized, e.g., according to the method disclosed in U.S. Pat. No. 6,635,618 B2, which is incorporated herein by reference.

### Examples

### Example 1

### Synthesis of N-Protected Decylaminoethanal (V)

Decylamine (11.4 g, 1 eq) was dissolved in 100 ml of BuOH (10 vol.) and 12.5 g of glyoxal 1,1-dimethylacetal (1 eq.) (60% solution in water) was added. The resulting mixture was distilled under vacuum at 60° C. to a volume residue of about 30 ml. Then 100 ml of BuOH was added and this resulting mixture was distilled again to reduce the volume to about 30 ml. After the second distillation the residual solution was analyzed by GC. Found: decylimminoethanolacetal: 98.64% (Area %).

Pt/C (1 g) was added to the volume-reduced solution prepared above, and the resulting mixture was kept under hydrogen atmosphere (3 atm) at 50° C. for 16 h. The reaction was checked by HPLC after 16 h. Found: Decylamminoethanal dimethylacetal (III): 97.58% (Area %). When the reaction was complete, the catalyst was filtered and the filtrate was distilled under vacuum at 60° C. to produce an oily residue. The residue was diluted with 200 ml of CH₂Cl₂ and cooled to +5° C. Diisopropylethylamine (11 g, 1.2 eq.) and benzylchloroformate (13.16 g, 1.1 eq.) were added to the solution and the resulting mixture was stirred at 5° C. for 2 h and then for another 3 h at room temperature.

The solution was then washed with 100 ml of water. The organic layer was then distilled under vacuum at 35° C. to form an oily residue. The residue was diluted with 50 ml of acetone and treated with 10 ml of 1M HCl for 1 h at room temperature. When this the reaction was complete, the two phases are separated. The upper phase was distilled under vacuum at 35° C. to produce an oil 24 g). Isolated yield: 85.81%. GC purity of N-Protected decylaminoethanal (V): 92.4%

### Example 2

### Synthesis of Decylaminoethanol (IIIa)

Decanal (30 g) was reacted with 47.5 g of ethanolamine (4 eq.) in 200 ml of MeOH (7 vol.) in an autoclave reactor under H₂ (3 atm) in the presence of 0.6 g of Pt/C (2% w/w) at room temperature for 16 h. After 16 h the catalyst was filtered and the panel washed with 50 ml of MeOH. The filtrate was distilled under vacuum at 35-40° C. to form an oil. The oil was diluted with 200 ml of water and the resulting mixture was extracted twice with 100 ml of CH₂Cl₂. The combined organic layers were distilled under vacuum at 35° C. to form an oil residue (33 g).

### Example 3

### Preparation of Decylaminoethanal Dimethylacetal (III)

Decanal (16.3 g) was mixed with 11 g of aminoacetaldehyde dimethylacetal in 50 ml of butanol. The solvent was distilled under vacuum at 60° C. to form an oily residue. The residual oil was diluted with 50 ml of butanol and distilled again to form an oily residue. The residual oil was diluted with 50 ml of BuOH, and then 1 g of Pt/C (50% water wet) was added. The resulting suspension was stirred at 50° C. under hydrogen atmosphere (3 atm) for 2 days. After 2 days Decylaminoethanal dimethylacetal (III) was obtained in 67% of yield.

### Example 4

### Preparation of Decylaminoethanal Dimethylacetal III

A mixture of glyoxal 1,1-dimethylacetal (18.6 g, 107 mmol), decylamine (15.0 g, 95.4 mmol) and 5% Pd/C (0.15 g) in methanol (100 ml) was hydrogenated at 50° C./5 bar for 3.5 h. The reaction mixture was then filtered through diatomaceous earth, and the apparatus and the filter cake were washed with MeOH (50 ml). The combined filtrate was evaporated under reduced pressure with two codistillations with toluene at 50° C. (90.6 mmol, Yield: 95%, purity: 97%).

### Example 5

### Preparation of Fmoc-Protected Decylaminoethanal Dimethylacetal IV from Decylaminoethanal Dimethylacetal III

A solution of decylaminoethanal dimethylacetal III (90.6 mmol) in toluene (180 ml) was cooled to 0-5° C. NaOH (1M, 109 ml, 1.2 eq.) was added, and the resulting mixture was stirred 5-10 min. A solution of Fmoc-Cl (23.4 g, 90.6 mmol) in toluene (90 ml) was then added dropwise over 15 min. The resulting mixture was stirred for 40 min at 0-5° C. in a cooling bath. The cooling bath was then removed and the mixture was allowed to warm to room temperature over 20 min. The water layer was removed, and the organic layer was washed with 20% aqueous NaCl (120 ml). The organic layer was then evaporated to dryness to afford a yellow syrup (81.5 mmol, Yield: 90%).

### Example 6

### Preparation of Crude N-Fmoc-Protected Decylaminoethanal V

HCOOH (163 ml) was added to the residue of Fmoc-protected decylaminoethanal dimethylacetal IV (81.5 mmol), and the mixture was stirred for 4 h at room temperature. The reaction mixture was then partitioned between n-heptane (326 ml) and 20% aqueous NaCl (300 ml). The organic layer was separated, washed with 20% aqueous NaCl (300 ml) and then with 7% aqueous NaHCO₃ (300 ml) and then was evaporated to dryness to give the Fmoc-protected decylaminoethanal V (73 mmol, Yield: 90%).

### Example 7

### Preparation of Imidazolidin Derivative V′.

HCOOH (163 ml) was added to the residue of Fmoc-protected decylaminoethanal dimethylacetal IV (81.5 mmol), and the mixture was stirred for 4 h at room temperature. The reaction mixture was then partitioned between n-heptane (360 ml) and 20% aqueous NaCl (300 ml). The organic layer was separated, washed with 20% aqueous NaCl (300 ml) and 7% aqueous NaHCO₃ (300 ml), dried with MgSO₄, and filtered. The filtrate was diluted with n-heptane (180 ml). 1,2-dianilinoethane (15.5 g, 73 mmol) was added and the resulting mixture was stirred for 30 h at room temperature and then for 5 h at 0° C. The product precipitated and was filtered off, washed with acetone (2×) and dried (20.5 g, Yield: 46%).

### Example 8

### Preparation of Imidazolidin Derivative V′

HCOOH (163 ml) was added to the residue of Fmoc-protected decylaminoethanal dimethylacetal IV (81.5 mmol) and the resulting mixture was stirred for 4 h at room temperature. The mixture was then partitioned between toluene (300 ml) and 20% aqueous NaCl (300 ml). The organic layer was separated, washed with 20% aqueous NaCl (300 ml) and then with 7% aqueous NaHCO₃ (300 ml), dried with MgSO4 and filtered. 1,2-Dianilinoethane (15.5 g, 73 mmol) was added and the resulting mixture was stirred for 1 h. The resulting solution was evaporated to dryness by one co-distillation with n-heptane. n-Heptane (440 ml) was added and the reaction mixture was stirred overnight at room temperature and at 0-5° C. for 5 h. The precipitated product was filtered off, washed with acetone and dried (27.1 g, Yield: 60%).

### Example 9

### Preparation of Aldehyde V from Imidazolidin Derivative V′.

Imidazolidine V′ (20.3 g, 33 mmol) was dissolved in THF (330 ml) at 30-40° C. The solution was cooled to 15-20° C. and celite (diatomaceous earth) (16.5 g) was added. A solution of p-toluenesulfonic acid (18.8 g, 99 mmol) in THF (83 ml) was added to the stirred suspension over 10 min at 15-20° C. The reaction mixture was stirred for 20 min, diluted with n-heptane (413 ml), stirred for 30 min and filtered. The filtrate was washed with concentrated NaHCO₃ (330 ml) and then with H₂O (165 ml) and then was evaporated to dryness (Yield: 90-95%).

### Example 10

### Preparation of p-toluenesulfonic Salt of Decylaminoethanal Dimethylacetal III′

p-Toluenesulfonic acid (5.03 g, 26.4 mmol) was dissolved in a solution containing 26.7 mmol of decylaminoethanal dimethylacetal III in toluene (27 ml) and the solution was evaporated under reduced pressure at 40-50° C., with one co-distillation with n-heptane (20 ml) to form a residue. N-Heptane (80 ml) was added to the residue and the mixture was stirred at room temperature for 2 h. The product precipitated and was filtered off, washed with n-heptane (2×) and dried (10.9 g, Yield: 98%).

### Example 11

### Preparation of Fmoc-Protected Decylaminoethanal Dimethylacetal IV from p-toluenesulfonic Salt of Decylaminoethanal Dimethylacetal III′

A suspension of p-toluenesulfonic salt of decylaminoethanal dimethylacetal III′ (5.0 g, 12 mmol) in toluene (20 ml) was cooled to 0-5° C. NaOH (1M, 28.8 ml, 2.4 eq.) was added dropwise over 10-15 min followed by addition of a solution of Fmoc-Cl (3.17 g, 12.2 mmol) in toluene (16 ml) in 15 min. The resulting mixture was stirred for 40 min at 0-5° C. in a cooling bath. The cooling bath was removed and the mixture was allowed to warm to room temperature during 20 min. The water layer was removed, and the organic layer was washed with 20% aqueous NaCl (2×20 ml) and then evaporated to dryness (10.8 mmol, Yield: 90%).

### Example 12

### Preparation of Telavancin from N-Fmoc-Protected Decylaminoethanal V

N-Fmoc-protected decylaminoethanal V prepared in Examples 6 or 9 is converted to televancin as described in U.S. Pat. No. 6,635,618, Example 2 (c)-(f) (col. 41, I. 52 to col. 42, I. 49).

What is claimed is:

1. Decylaminoethanal dialkyl acetal of formula III: 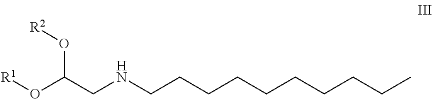
wherein R1 and R2 are independently selected from C1-C3 alkyl, or combined to form a 5- or 6-member cyclic acetal ring.

2. N-protected-decylaminoethanal dialkyl acetal of formula IV 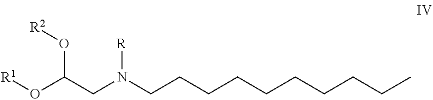
wherein R is an amine protecting group, R1 and R2 are independently selected from C1-C3 alkyl, or combined to form a 5- or 6-member cyclic acetal ring.

3. Imidazolidine derivative of formula V′ 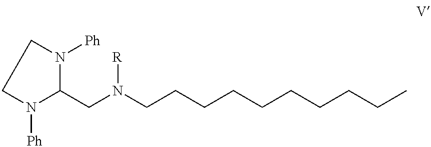
wherein R is an amine protecting group.

4. A process for preparing the decylaminoethanal dialkyl acetal of claim 1 comprising reacting:
i) glyoxal 1,1 dialkyl acetal of the following formula 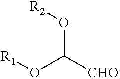
and decylamine, or
ii) aminoacetaldehyde dialkyl acetal of the following formula 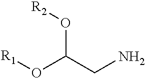
and decanal
to provide an imine intermediate; and
reducing the imine intermediate with a reducing agent to form the compound of formula III.

5. The process of claim 4, wherein the process is done in a one pot manner.

6. The process of claim 4, wherein the reducing agent is selected from the group consisting of sodium cyanoborohydride, sodium triacetoxyborohydride, pyridine/borane, sodium borohydride, zinc borohydride and molecular hydrogen in the presence of a hydrogenation catalyst.

7. The process of claim 6, wherein the hydrogenation catalyst is selected from the group consisting of platinum, platinum on carbon, palladium on carbon, or nickel.

8. A process for preparing Telavancin comprising providing decylaminoethanal dialkyl acetal of formula III and converting it to Telavancin.

9. The process of claim 8, wherein the step of providing the compound of formula III comprises the step of preparing the compound of formula III.

10. The process of claim 9, wherein the step of preparing the compound of formula III comprises:
reacting
i) glyoxal 1,1 dialkyl acetal of the following formula 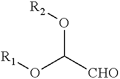
and decylamine, or
ii) aminoacetaldehyde dialkyl acetal of the following formula 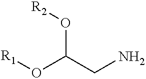
and decanal, to provide an imine intermediate; and
reducing the imine intermediate with a reducing agent to form the compound of formula III.

11. A process for preparing the N-protected-decylaminoethanal dialkyl acetal of claim 2 comprising reacting the compound of formula III 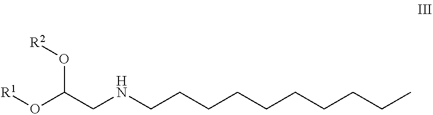
with an amine protecting group donor to form the N-protected-decylaminoethanal dialkyl acetal,
wherein R1 and R2 are independently selected from C1-C3 alkyl, or combined to form a 5- or 6-member cyclic acetal ring.

12. A process for preparing Telavancin comprising providing N-protected-decylaminoethanal dialkyl acetal of formula IV and converting it to Telavancin.

13. The process of claim 12, wherein the step of providing the compound of formula IV comprises the step of preparing the compound of formula IV.

14. The process of claim 13, wherein the step of preparing the compound of formula IV comprises:
reacting the compound of formula III 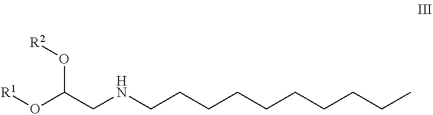
with an amine protecting group donor to form the N-protected-decylaminoethanal dialkyl acetal,
wherein R1 and R2 are independently selected from C1-C3 alkyl, or combined to form a 5- or 6-member cyclic acetal ring.

15. A process for preparing N-protected-decylaminoethanal of formula V 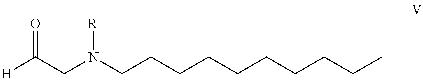
comprising reacting the N-protected-decylaminoethanal dialkyl acetal of claim 2 with an acid.

16. A process for preparing Telavancin comprising preparing N-protected-decylaminoethanal of formula V according to claim 15, and converting it to Telavancin.

17. A process for preparing an imidazolidine compound of formula V′ 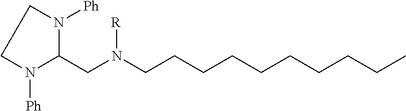
comprising
a) combining the N-protected-decylaminoethanal dialkyl acetal of formula IV 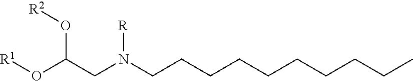
with an acid;
b) combining the result of step (a) with a solvent and water to obtain a two-phase mixture;
c) separating the organic layer of the mixture obtained in step (b);
d) combining the separated organic layer with 1,2 dianilinoethane to obtain a solution; and
e) precipitating an imidazolidine compound of formula V′ 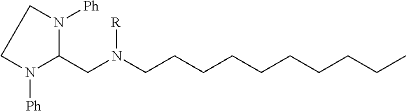
from the solution,
wherein R is an amine protecting group, R1 and R2 are independently selected from C1-C3 alkyl, or combined to form a 5- or 6-member cyclic acetal ring.

18. A process for preparing the compound of formula V 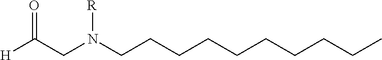
comprising preparing the compound of formula V′ 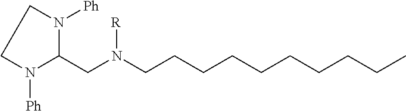
according to claim 17 and converting it to the compound of formula V,
wherein R is an amine protecting group.

19. A process for preparing Telavancin comprising providing the compound of formula V′ 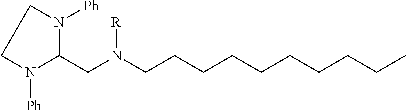
and converting it to Telavancin,
wherein R is an amine protecting group.

20. The process of claim 19, wherein the step of providing the compound of formula V′ comprises the step of preparing the compound of formula V′.

21. The process of claim 20, wherein the step of preparing the compound of formula V′ comprises:
a) combining the N-protected-decylaminoethanal dialkyl acetal of formula IV 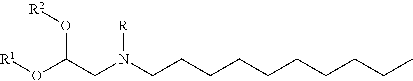
with an acid;
b) combining the result of step (a) with a solvent and water to obtain a two-phase mixture;
c) separating the organic layer of the mixture obtained in step (b);
d) combining the separated organic layer with 1,2 dianilinoethane to obtain a solution; and
e) precipitating an imidazolidine compound of formula V′ 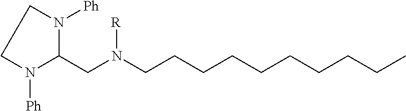
from the solution,
wherein R is an amine protecting group, R1 and R2 are independently selected from C1-C3 alkyl, or combined to form a 5- or 6-member cyclic acetal ring.

22. A process for preparing N-protected-decylaminoethanal of formula V 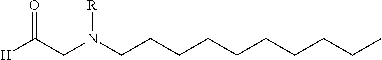
comprising:
a) reacting
i) glyoxal 1,1-dialkyl acetal 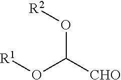
and decylamine, or
ii) aminoacetaldehyde dialkyl acetal 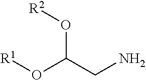
and decanal,
to provide an imine intermediate; and reducing the imine intermediate with a reducing agent to obtain decylaminoethanal dialkyl acetal of formula III, 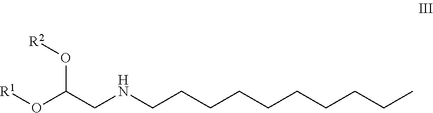
b) reacting the compound of formula III with an amine protecting group donor to obtain N-protected-decylaminoethanal dialkyl acetal of formula IV, and 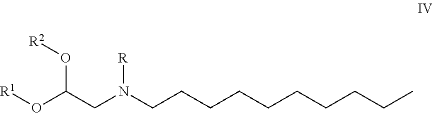
c) reacting the N-protected-decylaminoethanal dialkyl acetal of formula IV with an acid to obtain N-protected-decylaminoethanal of formula V 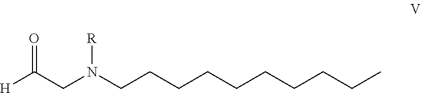
wherein R is an amine protecting group and R1 and R2 are independently selected from C1-C3 alkyl, or combined to form a 5- or 6-member cyclic acetal ring.

23. A process for preparing Telavancin comprising preparing N-protected-decylaminoethanal of formula V 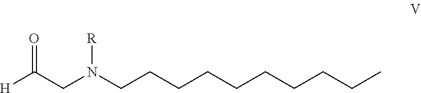
according to claim 22 and converting it to Telavancin,
wherein R is an amine protecting group.
